(19) United States
(12) Patent Application Publication
(10) Pub. No.: US 2021/0257068 A1
(43) Pub. Date: Aug. 19, 2021

(54) Universal wireless medical access card, UW-MaC (Biometric Digital Wireless ID Card)

(71) Applicant: Emmanuel Clarke, Winston Salem, NC (US)

(72) Inventor: Emmanuel Clarke, Winston Salem, NC (US)

(21) Appl. No.: 16/873,165

(22) Filed: Feb. 18, 2020

(51) Int. Cl. G16H 10/65; G06F 21/32; G06F 21/62; G06K 19/07; G06K 19/077; H04W 12/00; H04W 4/80

(52) U.S. Cl. CPC G16H 10/65; G06F 21/32; G06F 21/6245; H04W 4/80; G06K 19/07707; H04W 12/003; G06K 19/0718

(57) ABSTRACT

The device called Universal Wireless Medical Access Card, UW-MaC (a Biometric Digital Wireless ID Card), is a secured and smart ID card system that can store a person's medical records on an encrypted 16 Gigabits flash memory and can translate them in 100 different languages. The card comes secured with 256 bits of AES, SHA, and RSA encryption. The card/device is powered by a Coin-Shape lithium rechargeable battery that is constantly charged by an onboard thin film solar panel charging system.

### FIELD

The present application relates to a biometric digital electronic wireless medical device called (Universal Wireless Medical Access Card, UW-MaC (pronounced ‘all mac’, ‘you mac’ or ‘u mac’)) that can store a patient's medical record for access by any authorized medical practitioner in the event of a medical emergency or when it is needed. The card comes encrypted with 256 bits of AES and SHA encryption. The application that powers the device can translate the patient's medical records in 100 different languages.

### BACKGROUND

A portable electronic device the size of a credit card containing a person medical records or their Xray/MRI/CT Scan images, lab results that can be carried in a purse or the pocket can make the difference in how quickly medical practitioners can gain access to medical information and deliver urgent care to a sick person on demand. Currently in the United State, and in many third world countries around the world, there is no centralized electronic medical records system that can store patients' medical records electronically on an encrypted 16 Gigabits flash memory within a Bluetooth-enabled device for access by doctors and other medical practitioners in the absence of a complex computer network and Internet connection.

Quite recently, there has been so much buzz in the news about universal medical records system, but the truth is, there will never be such a thing like universal medical records system because hospitals will never allow a none-allied hospital to access their patients' medical records, let alone grant access to hospitals in other regions or in another country. This Bluetooth enabled device can connect wirelessly with an authorize medical practitioners' mobile phone, iPad, and computer, during a hospital or clinic visit without the need of a complex computer network or active Internet connection. This simple to use device will prevent most preventable deaths that are caused by wrongful medication or wrongful diagnosis because the card will give doctors and other medical practitioners instant access to a patient's medical records wherever they are. It will save patients and insurance companies a lot of money for unnecessary tests like CT Scan, MRI, Xray, Ultrasound etc., during a visit to a specialist for specialized treatment.

Using this biometric electronic digital wireless id card (UW-MaC) a patient will be able to carry their own medical history “On-The-Go”. For example, in the US if a person travels out of state and got sick and is taken to a nearby hospital, doctors at that hospital have no access to their medical records. This delays the delivery of care to that individual. With a portable device like the UW-MaC, the doctors will be able to access the patient's medical history the moment the patient walks in. This is the case with many third world and developing countries around the world, if a patient left from the village or a rural area clinic for a hospital in the city, that patient has to establish a new medical history at that hospital. Even at that, if that patient leaves that hospital for another hospital in the city, their medical records never go with them. But, with the UW-MaC, a patient can simply take their medical records with them on a credit-size electronic digital wireless id card from one doctor to another.

This unique device will be a lifesaver for medical patients living in developed as well as in many third-world countries and in many developing nations around the world. Doctor will be able to access a patient's record in any remote place that does not have Internet connection or a complex computer network. One of the unique abilities of the UW-MaC card system is its multi-language translation capability. The device/card (UW-MaC) will be able to speak many major languages found around the world due to its forward and backward language translation ability. The application (‘Luah Medical Mobile Application’) that powers the card, can translate records in 100 different languages.

For me as a computer engineer, this invention is very personal. My mother and my brother, both of whom were diabetics died at the ELWA hospital in 2005 (brother) and 2017 (mom) in Liberia when they fell sick and were taken to that hospital to seek an emergency medical treatment because they were running temperature with high fever. At the hospital, my brother was administered an Intravenous fluid or IV (drips) mostly glucose based and he died a day later after going into the diabetic coma. My mother also met a similar fate at the same hospital twelve years later. According to the incident report for my mother, the attending nurses did not know she was diabetic because they did not have access to her medical histories; they did what they normally do to most of their patients (pop a bag of fluid or drips into a patient's body) and wait for the fever to come down. The same thing happened to my Uncle, J. Archibald Brown in 2007 and quite recently a very good friend of mine, Coco died in 2018. All of those people died of preventable deaths after being wrongly medicated. Thousands of people around the world are dying every second in various hospitals and clinics because of the lack of knowledge on the part of caregivers. During critical emergency incident, access to a patient's medical records is very import to expedite the delivery of care to that patient.

Also, there is currently no way many Americans can instantly renew their driver's licenses from the comfort of their homes without contacting their states and local DMV's offices. The smart biometric identification card version of the UW-MaC can provide that option for many drivers' license holders around the world to instantly renew their license.

### SUMMARY

By allowing medical practitioners to have access to a patient's medical records in the event of a medical emergency is very critical to saving a patient's life. Having a portable device, the size of a credit card that can securely store a patient's medical records and give instant access to medical practitioners as well as the patient when they need it especially in a life and death situation is more than vital.

The following disclosed exemplary embodiment called Universal Wireless Medical Access Card, UW-MaC shows the internal components of the device that can store a patient's medical records on the 256 bits of encrypted device.

A second embodiment reveals the major input/output interactive features of the device.

In the third embodiment, an expanded front and the back-housing view of the device is shown. In this embodiment, an image of a finger is seeing on the screen of the device interacting with the fingerprint reader/optical sensor that captures the owner/user's fingerprint for authentication and unlocking the device before pairing with a Bluetooth enabled device that is running the device mobile application called “Luah”. The back housing of the device reveals the vent that allows heat to be disperse from the device internal processor.

In a fourth embodiment, the device's LCD screen reveal the picture and credential of the device's owner/user once the user's fingerprint has been captured and authenticated by the device internal biometric security system. The card's fingerprint reader is used to authenticate its owner's identity during a doctor's visit.

The fifth embodiment shows the device being paired with a smartphone that is running the card/device mobile application called, “Luah”. Without the mobile app. the device cannot be paired with any other Bluetooth enabled device.

The sixth embodiment shows the smartphone after being paired with the card/device. The Luah medical mobile application that is running on the phone gives options to a medical practitioner once it has been paired with a patient/card holder's device or card. This is done without physical contact between the medical practitioner and the patient/card owner.

In the seventh embodiment, the paired devices are seen exchanging data during a hospital visit. Based on the option made by the medical practitioner, the card allows the medical practitioner to read from and write to the device/card. The practitioner can sync the card to the smartphone or transfer records from the card onto the phone and from the phone back to the card for safe keeping.

The eight embodiment reveals how the Luah mobile application allows an authorization medical practitioner to write/safe or transfer updated medical records onto the card after medical practitioner has treated the patient and card's owner. At the end of a hospital visit, the medical practitioner can update the device with new information by transferring the updated information onto the card.

Further objects, feature, advantage and unique properties of device according to the present application become apparent from the detail description

### BRIEF DESCRIPTION OF THE DRAWINGS

In the following detailed of the current description, the teaching of the current application will be explained with reference to the example embodiments showing in the drawing in which:

FIG. 1-1 is a vertical architectural layout view of a microcontroller on a circuit board according to the first embodiment,

FIG. 1-2 is a vertical view of a device according to a second embodiment,

FIG. 1-3 is a vertical view of a device according to a third embodiment,

FIG. 1-4 is a vertical view of a device according to a fourth embodiment,

FIG. 1-5 is a vertical view of a device according to a fifth embodiment,

FIG. 1-6 is a vertical view of a device according to a sixth embodiment,

FIG. 1-7 is a vertical view of a device according to a seventh embodiment,

FIG. 1-8 is a vertical view of a device according to an eighth embodiment,

FIG. 1-9 is a vertical view of a device according to a ninth embodiment,

FIG. 1-10 is a vertical view of a device according to a tenth embodiment,

FIG. 1-11 is a vertical view of a device according to an eleventh embodiment,

FIG. 1-12 is a vertical view of a device according to a twelfth embodiment,

FIG. 1-13 is a vertical view of a device according to a thirteenth embodiment,

FIG. 1-14 is a vertical view of a device according to a fourteenth embodiment,

FIG. 1-15 is a vertical view of a device according to a fifteenth embodiment,

FIG. 1-16 is a vertical view of a device according to a sixteenth embodiment,

FIG. 1-17 is a vertical view of a device according to a seventeenth embodiment,

FIG. 1-18 is a vertical view of a device according to an eighteenth embodiment,

FIG. 1-19 is a vertical view of a device according to a nineteenth embodiment,

FIG. 1-20 is a vertical view of a device according to a twentieth embodiment, and

FIG. 1-21 is a vertical view of a device according to a twenty first embodiment.

### DETAILED DESCRIPTION

In the following detailed description, the device according to the teachings for this application in the form of a biometric digital electronic wireless identification card will be described by the embodiments. It should be noted that although only a medical ID card is described, the teachings of this application can also be used in other electronic identification device such as driver's license, insurance ID card, employee ID card, school ID card, TSA Security ID Card, government issued ID card, and many others identification that will offer access to someone's personal information.

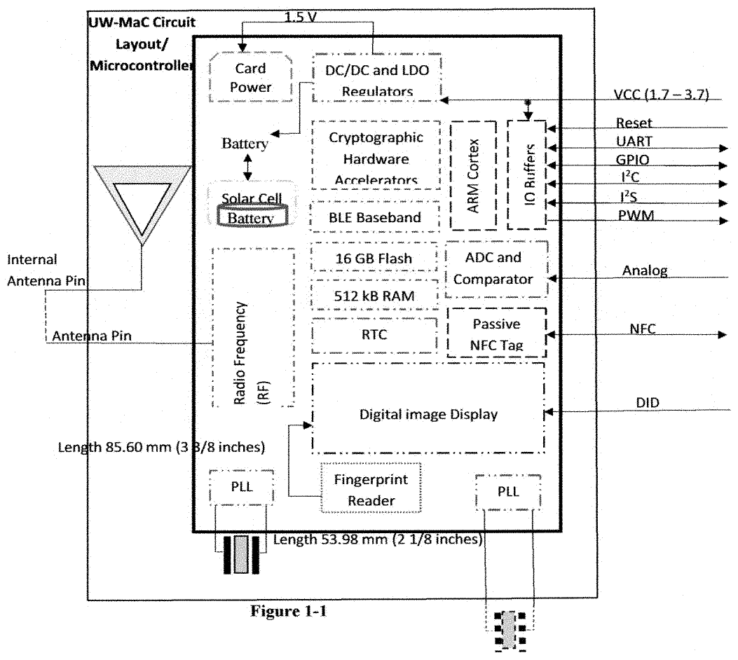

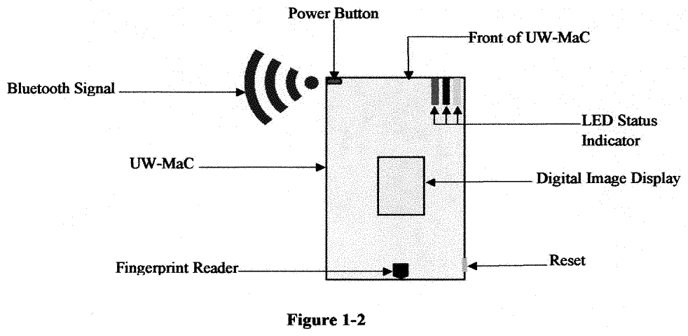

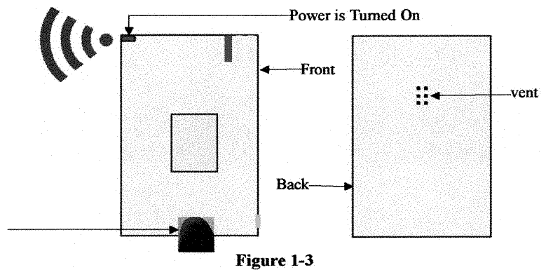

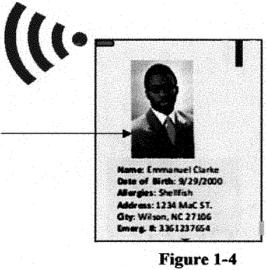

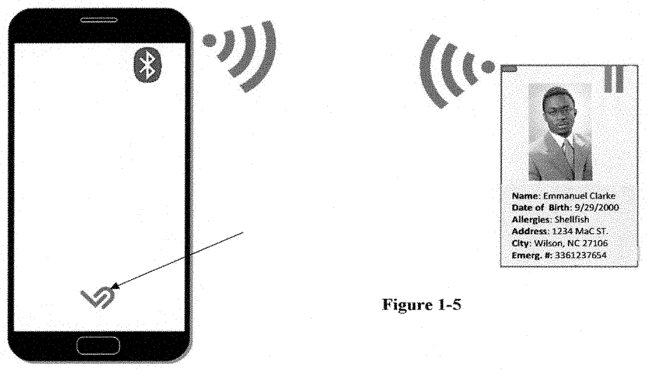

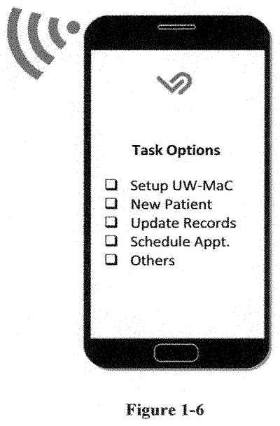

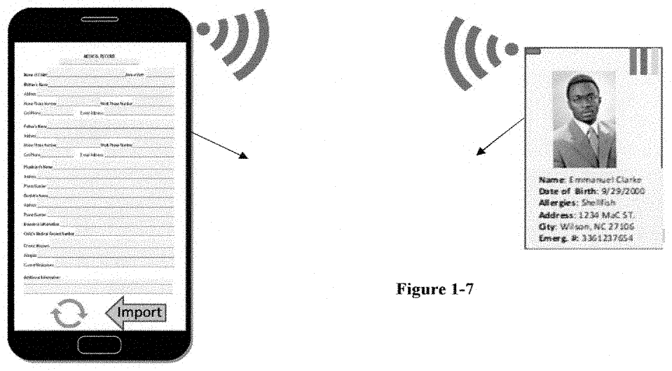

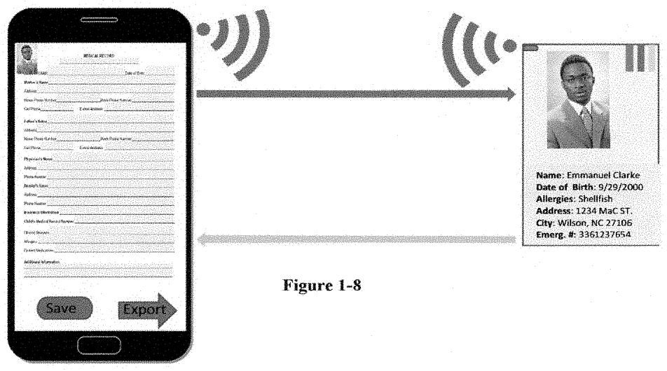

The first embodiment of the UW-Mac architecture is shown in FIG. 1-1. The embodiment in this figure shows the architectural layout of the UW-MaC Card System's (microcontroller) which contains the card power On/Off input pin or Voltage Common Connector (VCC), DC/DC and LDO Regulators that converts direct current on the circuit board of the microcontroller, battery (this battery is a rechargeable coin-shape batter that provide constant power for the card), solar cell recharges the battery for uninterrupted power supply for the card, ARM cortex (this is the main processor of the UW-MaC), BLE Baseband links the card to other Bluetooth enabled devices, Cryptographic Hardware Accelerators loads off the computing intensive tasks of the card, 16 GB Flash Memory card (this is the main memory of the card that stores data), 512 KB of RAM is a temporary storage that stores internal application of the card, Real Time Counter, RTC provides a generic low power timer on the low frequency clock source (LFCLK), IO Buffer is a temporary storage that stores data that passes between the UW-MaC and the device that it is paired with (it contains the GPIO, PMW, I²C, I²S, UART, Reset pin, and the VCC. Please refer to technical details section), ADC and Comparator serves as differential pairs for measuring the voltage of various input across the circuit board of the card system, Passive NFC Tag allows data to be read from or to the UW-MaC using an NFC reader, Fingerprint Reader captures user/owner fingerprint and compares it with system's stored fingerprint for user/owner's authentication, Antenna transmits radio frequency to a nearby Bluetooth enabled device that is in the range of 10 to 25 feet, Digital Image Display, DID is the main screen of the UW-MaC that displays owner's information and data for owner, PLL matches the frequency of an input signal.

In this second embodiment of the card/device as illustrated in FIG. 1-2. depicts the front and back rectangular housing unit that contains all of the electronic components of the device/card/UW-MaC, power button (this button turns the card on or off), front screen of UW-MaC contains the LCD of the card, Bluetooth signal internal antenna (the Bluetooth signal sends signals to other Bluetooth enabled devices within range for pairing and connection), LED status indicator (shows the status of the ID Card once it is turned on by user), digital image display (screen of card that displays the user's information and or medical records), reset button (this button restores card in case of a technical glitch), fingerprint reader captures card owner fingerprint for authentication and unlocking the card for data or information access by user or doctor during hospital visit.

In the third embodiment as revealed by FIG. 1-3 shows an extended view of the front screen and the back-housing are revealed. The illustration shows the device/card's front screen where a user/owner is scanning their fingerprint to unlock the device for pairing with a Bluetooth enabled device. The back-housing shows the vent where heat is dispersed from the device's processor. This allows the card to remain cool under 60 degrees Fahrenheit while it is being used or synced with a mobile device or computer running the Luah mobile application.

In this fourth embodiment of the card/device with an owner/user image and credential on the front screen as illustrated in FIG. 1-4. The picture of the device's owner/user is displayed on the device's screen once the device/card's internal security system has authenticated the scanned fingerprint as that of the owner. The device cannot be unlocked without the owner's biometric identity. This is the device's core security feature.

In the fifth embodiment we see the device being paired with the smartphone as shown in FIG. 1-5. The smartphone that is running the Luah mobile application must be initiated by tapping or clicking the app's icon. Once the app is running on the phone, it researched for the id card/device for pairing. Once the card/device is found, the app displays the card's name on the screen for the medical practitioner to click as a final step to pairing the two devices. This important step must be done in order for a medical practitioner to access the contents of the card/device or to import a patient's medical records from the card to the mobile phone. The step for pairing the id card/device is the same for iPad, Bluetooth enabled computer. The card/device also has a micro USB port that can allow a medical practitioner to access a patient's medical records in the event where the computer is not Bluetooth enabled. Either way, the card will still be synced to the computer.

The internal components, software, firmware, encryption technology, protocol and structure will not be described in detail since such technology is generally well known.

In the sixth embodiment as illustrated in FIG. 1-6, the application running on the smartphone gives the medical practitioner option after the card/device and the phone have been paired. Based on the selected choice, the application's screen will display that choice.

A seventh embodiment of the card/device as illustrated in FIG. 1-7, shows the smartphone displaying a new blank screen which is based on the option/choice selected by the medical practitioner after phone has been synced with the card/device. For example, if the medical practitioner selected new patient, a blank screen would appear so the practitioner can enter the new patient's information into the application which he would later save to the card/device and to the cloud.

In the eighth embodiment as illustrated in FIG. 1-8, the Luah Medical Mobile allows the medical practitioner to Export the patient's records to the card/device or Save it to the cloud for future access. This allow the patient to carry their updated medical records with them anywhere they go. In case the card gets missing, a patient can purchase a new card and their existing medical records that is saved to the cloud can be downloaded to the new card after it has been setup by an authorized medical practitioner. This two-factor storage reinforces data security.

The front display/screen and user-interface elements thereon can in an embodiment be made from scratch resistant materials, thereby rendering the front face even more resistant to being scratched.

The various aspect of what is described above can be used alone or in combinations. It should be noted that the teaching of this application is not limited to the use in medical records storage, but can be equally well applied to government identification and driver's license, students' id card, access control card, TSA travel access card, etc.

The teaching of the present application has numerous advantages. Different embodiments or implementations may yield one or more of the following advantages. It should be noted that this is not an exhaustive list and there may be other advantages which are not described herein. For example, one advantage of the teaching of this application is it provides for a device with an LCD screen that is less vulnerable to be scratch that displays the owner's picture and information. It is another advantage of the present invention that it provides end-to-end encryption for patient's data on both the card and the Luah mobile application. It is another advantage of the present invention that it provides for a device the size of a credit card and with the depth of a quarter that can easily fit in a person's wallet or purse and can easily be transported.

Although the teaching of the present application has been described in detail for purpose of illustration, it is understood that such detail is solely for that purpose, and variation can be made therein by the skilled in the art without departing from the scope of the teaching of this application. For example, the device has been illustrated with a housing that is substantially rectangular back, but it is understood that the back panel does not need to be rectangular, the outline of the housing could have any other suitable shape that is used in these kinds of devices. Further, the device has been illustrated with a housing that is in one piece, but it should be understood that the teaching can also be applied to devices that have detachable or several housing parts, such as known from back or front fold, and twist like a wallet.

For example, although the teaching of the present application has been described in terms of a biometric electronic digital wireless id card/device for storing patients' medical records, it should be appreciated that the teachings of the present application may also be applied to other types of electronic devices, such as biometric government issued identifications like US Green Card, driver's license, state's id card, students' id card, library cards, TSA Frequent Traveler id card, health insurance id card, and many others. It should also be noted that there are many alternative ways of implementing the methods and apparatuses of the teaching of the present application.

For the teaching of the present application to properly function as intended, the user must do the following:
1. The card/device must be powered on by the patient/owner
2. A doctor or an authorized user must pair their Bluetooth-enabled mobile device with the card/device. The card's barcode can also be scanned as a means of quickly accessing basic patient's medical record.
3. Patient must unlock card/device system scanning/using their fingerprint
4. Once unlocked, patient's picture will appear on LED screen of the device/card system

For the teaching of the present application for “Luah” to properly work, the following steps must be taken:
1. Download “Luah” Mobile Medical App from the Apple or Google Store to your Bluetooth-enable mobile device or computer (laptop or desktop)
2. Install and setup Luah by creating a profile for a user (doctor and medical practitioners only)
3. A One Time Password/OTP is sent to the user for authentication purpose
4. Tap or click the “LUAH” icon on the mobile device or computer to launch application
5. Add to or update patient's record (you can import an existing patient's record to mobile device)
6. Create patient's profile (new patient only)
7. Activate UW-MaC with Luah (new patient only)
   a. Add fingerprint (new patient only)
   b. Add and store picture (new patient only)
8. Once done, save and transfer record to UW-MaC card system.

This device/card system is a medical record “To Go” device that brings patients and doctors/medical practitioners both convenience and data security. This biometric electronic digital wireless device/card gives patients a full control over their medical records as to who can access it. In order for the device (UW-MaC) card system to work, the user/patient card must be turned on and within range (at least 25 to 50 feet) of the receiving Bluetooth-enable wireless device that is running the “Luah” mobile medical application. Once the UW-MaC is powered on, it can be detected by other Bluetooth-enabled wireless devices nearby, but it can only connect to a device running the “Luah” medical mobile application. The device requires no Internet connection to work once the application is downloaded from the Apple or Google Play Store. The device/card (UW-MaC) uses Low Energy Bluetooth (BLE) technology that allows the card to connect to a doctor's or a medical practitioner's mobile device or a Bluetooth-enabled computer. The device/card (UW-MaC) can transfer data up to 1 Mbps in a noiseless environment. The device/care (UW-MaC) will enable people living in remote parts of the world to have access to their own electronic medical records that can be access by any doctor or licensed medical practitioner using the ‘Luah” Medical Mobile Application on their mobile device anywhere they travel in the world irrespective of their language. The powerful language translator that is built within the LUAH Mobile Medical Application can translate a patient's medical record in any of the world's major languages. For example, the application can translate from English to French and back from French to English, etc. This powerful forward and reverse translation ability that is built within the application makes the device one of the most important piece of possessed-objects anyone can own, especially people living in underdeveloped and developing world without a centralized medical record keeping system or and a medical system that does not integrate with other nations’ healthcare systems.

Features described in the preceding description may be used in combination other than the combination explicitly described.

The invention claimed is:

1. A biometric electronic digital device with a rectangular front and back housing, wherein can store a patient's medical records wherein its mobile application can translate medical records in one hundred different languages.

2. A biometric electronic digital device according to claim 1, wherein uses optical sensor to capture owner/user's fingerprint as a way of authenticating the owner before unlocking the device for pairing with other Bluetooth-enabled devices running the device's proprietary medical mobile application called ‘Luah’ which powers the device.

3. A biometric electronic digital device according to claim 2, wherein the card's data is encrypted with 256 bits of AES and SHA end to end data encryption on the Luah application.

4. A biometric electronic digital device according to claim 1, wherein the device has an LCD image display for displaying the owner/user's picture and information after authentication.

5. A biometric electronic digital device according to claim 4, wherein the front LCD is embedded with the optical sensor/reader for capturing fingerprint for system authentication of the owner/user.

6. A biometric electronic digital device according to claim 1, wherein the device can pair/connect wirelessly via Bluetooth with other Bluetooth-enabled devices devices/smartphones/computers/iPad running the “Luah” Medical Mobile Application where the underlining application powers the card/device that enable card/device to connect/sync wirelessly with other Bluetooth-enable devices.

7. A biometric electronic digital device according to claim 6, wherein the card/device can interchange data with the other Bluetooth devices once paired.
